(19) United States
(12) Patent Application Publication
(10) Pub. No.: US 2023/0285118 A1
(43) Pub. Date: Sep. 14, 2023

(54) PROTECTION DEVICE FOR TRANSORAL SURGERY

(71) Applicant: SEOUL NATIONAL UNIVERSITY R&DB FOUNDATION, Seoul (KR)

(72) Inventors: Su-jin KIM, Seoul (KR); Hyoun-Joong KONG, Seoul (KR)

(21) Appl. No.: 18/246,810

(22) PCT Filed: Aug. 6, 2021
(86) PCT No.: PCT/KR2021/010378

§ 371 Date: Mar. 27, 2023

(30) Foreign Application Priority Data
Sep. 28, 2020 (KR) 10-2020-0125737

(51) Int. Cl. A61C 5/90; A61C 5/80

(52) U.S. Cl. CPC A61C 5/90; A61C 5/80; A61C 2201/00

(57) ABSTRACT

A protection device for transoral surgery includes: a main body configured to be fitted into an oral cavity of a human body and to expand a mouth vestibule between teeth and a lip of the human body; and at least one insertion device provided to the main body and having a through hole formed therethrough to enable insertion of a trocar required for a surgery. The main body includes: a tooth coupling part configured to be fitted on the teeth of the human body; an expansion part to be disposed on an outside of the tooth coupling part and configured to expand the mouth vestibule to allow the teeth and the lip to be spaced apart from each other; and an extension part configured to connect the tooth coupling part and the expansion part.

### TECHNICAL FIELD

The present disclosure relates to a protection device for transoral surgery. One of the most typical surgeries performed through the mouth is thyroid surgery. When performing thyroid surgery, injuries to incisional wounds, lacerations and ruptures of the lips, etc. may be caused. The present disclosure relates to a protection device for transoral thyroid surgery that can prevent this problem.

### BACKGROUND ART

The thyroid gland is an endocrine organ situated in front of the respiratory tract which serves as a passageway for air. The thyroid gland normally weighs about 16 grams in women and about 18 grams in men. Thyroxine hormones released from the thyroid gland are a major contributor in the regulation of metabolism in the body.

A nodule on the thyroid is a lesion that is distinct from the surrounding thyroid tissue, which can be seen as a “lump” and includes both malignant and benign tumors. If a thyroid nodule is suspicious for malignancy in clinical manifestations and ultrasonographic findings, fine needle aspiration cytology or core needle biopsy is performed, and thyroidectomy is recommended for malignancy to remove the thyroid by surgery. In the case of a benign nodule, thyroidectomy is recommended if it is large or symptomatic or tracheal deviation is present. Thyroidectomy is also performed on patients with uncontrolled hyperthyroidism.

Traditionally, the thyroid has been removed by surgery which involves removing a thyroid nodule through a neck incision that leaves a visible scar on the neck. In this regard, a variety of surgical approaches have been studied to reduce the size of wounds, and more recently, transoral surgery is developing fast. Transoral surgery is the most effective thyroid operation that makes the smallest incisions possible that has ever existed.

Transoral thyroid surgery is performed by making three incisions on the mucosa of the oral vestibule situated between the lower teeth and the lower lip and passing a trocar through the incisions through which a surgical instrument is inserted.

In this case, the narrow space in the oral vestibule may cause injury to incision sites in the oral vestibule due to movement of a surgical instrument or robotic equipment. Moreover, complications may be produced, such as ruptures of the corners of the lips of a patient.

### DETAILED DESCRIPTION OF INVENTION

### Technical Problem

Embodiments of the present disclosure have been devised based on the above-described background, and provide a protection device for transoral surgery which prevents injuries to incisional wounds or lacerations of incision sites and also prevents wounds such as ruptures of the corners of the lips.

### Means for Solving the Problems

According to one aspect of the present disclosure, a protection device for transoral surgery may include: a main body configured to be fitted into an oral cavity of a human body and to expand a mouth vestibule between teeth and a lip of the human body; and at least one insertion device provided to the main body and having a through hole formed therethrough to enable insertion of a trocar required for a surgery. The main body includes: a tooth coupling part configured to be fitted on the teeth of the human body; an expansion part to be disposed on an outside of the tooth coupling part and configured to expand the mouth vestibule to allow the teeth and the lip to be spaced apart from each other; and an extension part configured to connect the tooth coupling part and the expansion part.

The at least one insertion device may include a plurality of insertion devices, and one or more of the plurality of insertion devices are disposed on the outside of the tooth coupling part.

The at least one insertion device may include a plurality of insertion devices, and one of the plurality of insertion devices is disposed on an inside of the tooth coupling part.

The main body may further include a tooth row extension part having a predetermined width and formed on the inside of the tooth coupling part, and one of the plurality of insertion devices may be disposed in the tooth row extension part.

The main body may further include at least one elastic part having a predetermined width, which is formed of a softer material than a material of the tooth coupling part and has a predetermined level of elasticity, and the at least one insertion device may be disposed in the at least one elastic part, respectively.

The main body may further include: a first safety part and a second safety part which are placed on the expansion part, and formed of a rigid material that is harder than a material of the expansion part. The least one insertion device may be placed between the first safety part and the second safety part.

The at least one insertion device may include an insert. The insert may include: a cylindrical insertion housing having a hollow therein; and an insertion moving object having the through hole and disposed in the hollow of the insertion housing, at least a portion of the insertion moving object having a spherical shape so that an orientation of the through hole is changed.

A height of the insertion housing may be smaller than a diameter of the insertion moving object.

The at least one insertion device may further include: a moving plate that has a shape of a plate with a predetermine length, and has therein an insertion hole of a predetermined length formed so that the insert moves in a lengthwise direction of the plate shape

The insertion hole formed in the moving plate may include: a plurality of fixing hole parts of a predetermined width so as to fix the insert; and a moving hole part that extends between the plurality of fixing hole parts and is smaller in width than the fixing hole parts.

The protection device for transoral surgery may further include: at least one elastic part whose planar shape is elliptical, and which is formed of a softer material than a material of the tooth coupling part and has a predetermined level of elasticity. The at least one insertion device may be placed in the at least one elastic part, respectively, so that the lengthwise direction of the moving plate is parallel to a minor axis direction of the elliptical shape of the at least one elastic part.

### Effect of Invention

According to embodiments of the present disclosure, a surgical instrument or robotic equipment may be kept from making contact with other areas in the oral cavity of a patient because the protection device has an extension part and an expansion part in regions other than incision sites in the oral vestibule into which a trocar is inserted when performing transoral surgery.

Furthermore, it is possible to secure a space for a surgical instrument or robotic equipment to move within it because the expanded portion of the protection device holds the patient’s lips so that the lower lip and the opposite corners of the lips are kept apart, and therefore to prevent injuries to the corners of the lips, such as lacerations or ruptures, due to movement of the surgical instrument or robotic equipment.

### BRIEF DESCRIPTION OF DRAWINGS

FIG. 1 is a view illustrating a region to be incised for transoral surgery according to one embodiment of the present disclosure.

FIG. 2 is a view illustrating a protection device for transoral surgery according to the embodiment of the present disclosure.

FIG. 3 is a view illustrating an insertion device of the protection device for transoral surgery according to the embodiment of the present disclosure.

FIG. 4 is a view illustrating an insert of the protection device for transoral surgery according to the embodiment of the present disclosure.

FIG. 5 is a view illustrating an elastic part of the protection device for transoral surgery according to the embodiment of the present disclosure.

FIG. 6 is a view illustrating a modification of an insertion device of the protection device for transoral surgery according to the embodiment of the present disclosure.

FIG. 7 is a view illustrating another modification of the insertion device of the protection device for transoral surgery according to the embodiment of the present disclosure.

FIG. 8 is a view illustrating a region to be incised for transoral surgery according to another embodiment of the present disclosure.

FIG. 9 is a view illustrating a protection device for transoral surgery according to another embodiment of the present disclosure.

### EMBODIMENTS OF INVENTION

Hereinafter, specific embodiments for implementing a spirit of the present disclosure will be described in detail with reference to the drawings.

In describing the present disclosure, detailed descriptions of well-known functions or configurations related to the present disclosure will be omitted when it is deemed that they may unnecessarily obscure the subject matter of the present disclosure.

When an element is referred to as being ‘connected’ to, ‘supported’ by, ‘accessed’ to, ‘supplied’ to, ‘transferred’ to, or ‘contacted’ with another element, it should be understood that the element may be directly connected to, supported by, accessed to, supplied to, transferred to, or contacted with another element, but that other elements may exist in the middle.

The terms used in the present disclosure are only used for describing specific embodiments, and are not intended to limit the present disclosure. Singular expressions include plural expressions unless the context clearly indicates otherwise.

Further, in the present disclosure, it is to be noted that expressions, such as the upper side, the lower side, and side surface are described based on the illustration of drawings, but may be modified if directions of corresponding objects are changed. For the same reasons, some components are exaggerated, omitted, or schematically illustrated in the accompanying drawings, and the size of each component does not fully reflect the actual size.

Terms including ordinal numbers, such as first and second, may be used for describing various elements, but the corresponding elements are not limited by these terms. These terms are only used for the purpose of distinguishing one element from another element.

In the present specification, it is to be understood that the terms such as “including” are intended to indicate the existence of the certain features, areas, integers, steps, actions, elements, combinations, and/or groups thereof disclosed in the specification, and are not intended to preclude the possibility that one or more other certain features, areas, integers, steps, actions, elements, combinations, and/or groups thereof may exist or may be added.

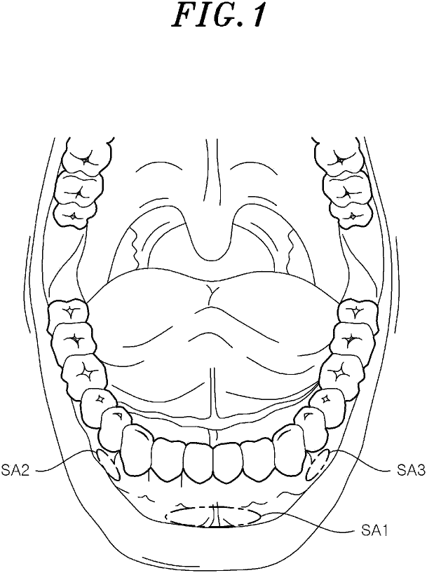

Referring to FIGS. 1 to 7, a protection device 10 for transoral surgery according to one embodiment of the present disclosure will be described. The protection device 10 may minimize non-surgical injuries to the mouth of a patient during transoral surgery by being fitted into the oral cavity of the patient. ~ An example of such transoral surgery is thyroid surgery which is performed through the mouth.

The protection device 10 for transoral surgery may include a main body 100, a first insertion device 210, a second insertion device 220, and a third insertion device 230.

The main body 100 may be fitted into the oral cavity of a patient and secure a surgical space for transoral surgery. To this end, the main body 100 may be made of a rigid material and have a certain level of elasticity. The main body 100 may include a tooth coupling part 110, an extension part 120, an expansion part 130, a first safety part 140, a second safety part 150, a first elastic part 160, a second elastic part 170, and a third elastic part 180.

The tooth coupling part 110 is provided to be coupled to the lower teeth of the patient so as to be stably fitted into the oral cavity of the patient. The tooth coupling part 110 may have a groove so as to be coupled to the lower teeth of the patient, and the lower row of teeth may be inserted into the groove formed in the tooth coupling part 110, thus causing the main body 100 to be coupled to the lower teeth.

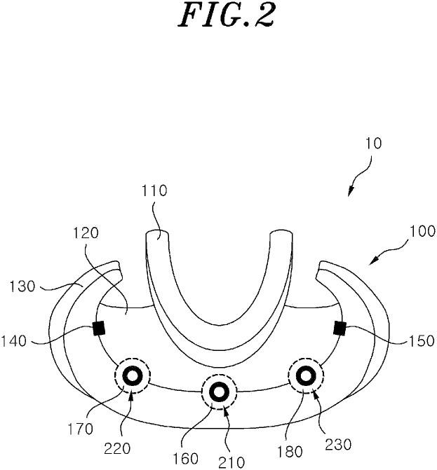

To this end, the tooth coupling part 110 may have a U shape so as to correspond to the shape of the lower row of teeth. FIG. 2 illustrates an upper surface of the tooth coupling part 110 but does not illustrate the groove of the tooth coupling part 110 formed on a back surface of the tooth coupling part 110.

The extension part 120 is placed on the outside of the tooth coupling part 110 having a U shape, and connects the tooth coupling part 110 and the expansion part 130. The extension part 120 may be formed to have a predetermined width between the tooth coupling part 110 and the expansion part 130. In other words, the tooth coupling part 110 and the expansion part 130 may be spaced apart from each other by as much as the width of the extension part 120. The back surface of the extension part 120 may make contact with the oral vestibule when the main body 100 is fitted into the oral cavity of the patient.

In this instance, when making contact with the oral vestibule, the extension part 120 may come into partial contact with the gum situated below the teeth, and may keep the lips of the patient apart from the rows of teeth. To this end, the extension part 120 may be made of a rigid material as in the tooth coupling part 110.

The expansion part 130 extends outward from the extension part 120. The expansion part 130, when fitted into the oral cavity of the patient, may be situated in an area of the oral vestibule that is adjacent to the lips. The expansion part 130 may have, but not limited to, a shape that covers part of the lips of the patient.

Once the main body 100 is fitted into the oral cavity of the patient, the expansion part 130 serves to keep the mouth open, as in a holder for opening the mouth. That is, the expansion part 130 may push the opposite corners of the lips outward to expand and open the mouth. Also, the expansion part 130 may have a shape that helps push the lower lip outward so as to expose the oral vestibule between the lower lip and the arrangement of the lower teeth. In other words, the expansion part 130 serves to expand the oral vestibule.

As illustrated in FIG. 2, the first safety part 140 and the second safety part 150 may be placed between the extension part 120 and the expansion part 130, symmetrically on the opposite sides of the extension part 120. The first safety part 140 and the second safety part 150 may be formed of a rigid material that is harder than the material of the main body 100. For example, if the tooth coupling part 110, extension part 120, and expansion part 130 constituting the main body 100 are made of hard silicon, rubber, or the like, the first safety part 140 and the second safety part 150 may be made of a metallic material or the like.

Accordingly, even when a trocar 300, a surgical instrument, or robotic equipment moves outside of the extension part 120 or the expansion 130 during transoral surgery, the first safety part 140 and the second safety part 150 may prevent the extension part 120 or the expansion part 130 from getting injured by the surgical instrument or the robotic equipment. Also, the first safety part 140 and the second safety part 140 may prevent the trocar 400, the surgical instrument, or the robotic equipment from passing through the main body 100 and causing injury to the oral vestibule or lips of the patient.

The first elastic part 160, the second elastic part 170, and the third elastic part 180 may be placed at one or both of the extension part 120 and the expansion part 130. The first elastic part 160, the second elastic part 170, and the third elastic part 180 may be formed of a softer material than the materials of the tooth coupling part 110, extension part 120, and expansion part 130, and may have a certain level of elasticity.

The first elastic part 160, the second elastic part 170, and the third elastic part 180 may have a substantially circular shape as illustrated in FIG. 2, and may have the first insertion device 210, the second insertion device 220, and the third insertion device 230 respectively on the inside.

Accordingly, the first insertion device 210, the second insertion device 220, and the third insertion device 230 may be moved a predetermined distance by the first elastic part 160, the second elastic part 170, and the third elastic part 180. That is, the first elastic part 160, the second elastic part 170, and the third elastic part 180 may be moved by an external force, which means that they may be moved a predetermined distance by the elasticity of the first elastic part 160, the second elastic part 170, and the third elastic part 180.

In the embodiment, the first elastic part 160 may be placed midway between the extension part 120 and the expansion part 130. That is, the first elastic part 160 may be placed at a position corresponding to a first incision area SA1 placed in front of the midline of the oral vestibule illustrated in FIG. 1.

The second elastic part 170 and the third elastic part 180 may be placed between the extension part 120 and the expansion part 130, and may be spaced apart from each other with the first elastic part 160 interposed therebetween to be disposed at positions corresponding to a second incision area SA2 and a third incision area SA3 which are placed adjacent to the opposite ends of the oral vestibule.

The first insertion device 210, the second insertion device 220, and the third insertion device 230 may have the same construction, and may be placed inside the first elastic part 160, the second elastic part 170, and the third elastic part 180, respectively. In this instance, the first insertion device 210, the second insertion device 220, and the third insertion device 230 may be placed integrally with the first elastic part 160, the second elastic part 170, and the third elastic part 180, respectively.

The first insertion device 210, the second insertion device 220, and the third insertion device 230 each may have a hole through which the trocar 300 can pass. Thus, once the main body 100 is placed in the oral cavity of a patient after the first incision area SA1, the second incision area SA2, and third incision area SA3 of the oral vestibule are incised for transoral surgery, the first insertion device 210, the second insertion device 220, and the third insertion device 230 may be placed at positions corresponding to the first incision area SA1, the second incision area SA2, and the third incision area SA3, respectively.

Accordingly, the trocar 300 may be inserted through each of the first insertion device 210, the second insertion device 220, and the third insertion device 230, allowing the trocar 300 to approach the first incision area SA1, second incision area SA2, and third incision area SA3.

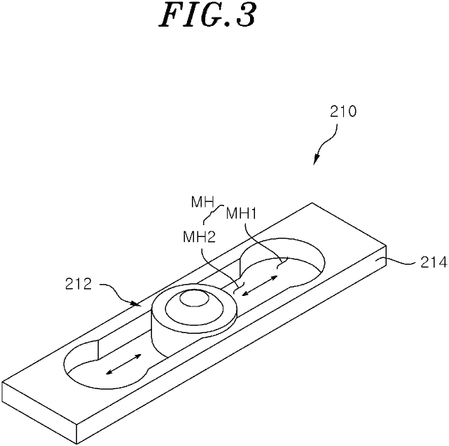

The first insertion device 210, the second insertion device 220, and the third insertion device 230 each may include an insert 212 and a moving plate 214. Referring to FIGS. 3 to 5, the first insertion device 210 will be described in more detail.

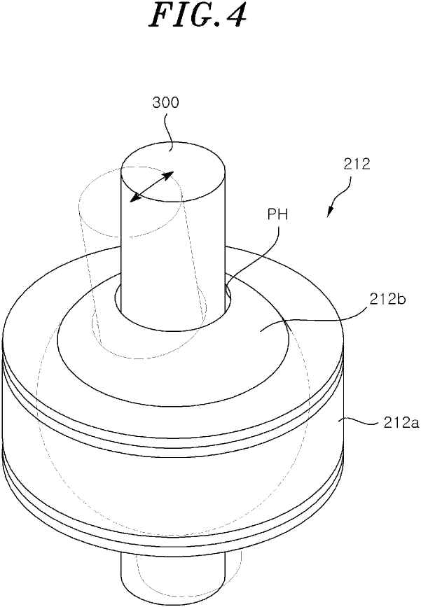

As illustrated in FIG. 4, the insert 212 has a through-hole PH through which the trocar 300 can pass. The angle of the trocar 300 may be adjusted while the trocar 300 is passed through the insert 212. To this end, the insert 212 may include an insertion housing 212a and an insertion moving object 212b.

The insertion housing 212a may have a substantially cylindrical shape and have a hollow so that the insertion moving object 212b is placed within it.

The insertion moving object 212b may be placed in the hollow of the insertion housing 212a and be spherically shaped. Also, the spherically-shaped insertion moving object 212b may have a through-hole PH through which the trocar 300 can pass. At least part of the insertion moving object 212b may be spherically shaped so that the orientation of the through-hole PH is changed. In other words, the direction in which the through-hole PH faces may be changed as the spherically-shaped insertion moving object 212b rotates in the hollow of the insertion housing 212a.

Accordingly, since the insertion moving object 212b has a spherical shape while the insertion moving object 212b is placed in the hollow of the insertion housing 212a, the insertion moving object 212b may rotate within a predetermined angle range within the insertion housing 212a. In this instance, the direction of rotation of the insertion moving object 212b may rotate in any direction since the insertion moving object 212b has a spherical shape.

Here, the diameter of the insertion housing 212a may be larger than the diameter of the insertion moving object 212b, and the height of the insertion housing 212a may be smaller than the diameter of the insertion moving object 212b. That is, the insertion moving object 212b may be placed in such a way as to protrude from the upper and lower ends of the insertion housing 212a.

The insertion moving object 212b may rotate in the insertion housing 212a since the angle of the trocar 300 is adjusted by the worker while the trocar 300 is passed through the through-hole PH. Accordingly, when the insertion moving object 212b rotates with the movement of the trocar 300 while the trocar 300 is placed so as to pass through the through-hole PH of the insertion moving object 212b, the insertion moving object 212b may rotate in the insertion housing 212a within a range in which the trocar object comes into contact with the insertion housing 212a.

As illustrated in FIG. 3, the moving plate 214 may have the shape of a rectangular plate and have an insertion hole MH on the inside.

As illustrated in the drawing, the insertion hole MH may be formed in such a way that the insert 212 moves in a lengthwise direction of the moving plate 214. In this instance, the insertion hole MH includes three fixing hole parts MH1 which are larger in width so as to fix the insert 212 and a moving hole part MH2 formed between the three fixing hole parts MH1 which is smaller in width so as to allow the insert 212 to move.

Accordingly, the worker is able to move the insert 212 to any one of the fixing hole parts MH1 along the moving hole part MH2, and then the insert 212 may be fixed in position to the fixing hole part MH1.

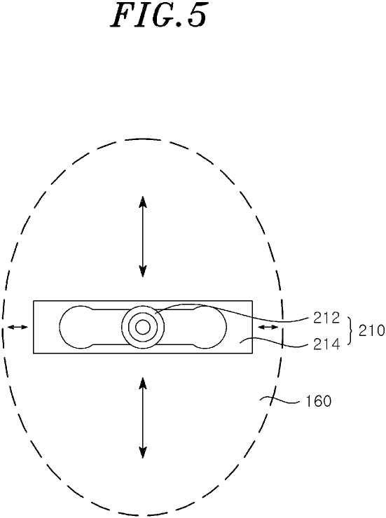

Moreover, in the embodiment, the first elastic part 160 is formed around the first insertion device 210 including the insert 212 and the moving plate 214, as illustrated in FIG. 2. In this instance, the first elastic part 160 may have, but not limited to, a circular shape, and also may have an elliptical shape, as illustrated in FIG. 5.

That is, when the first elastic part 160 has an elliptical shape as illustrated in FIG. 5, the lengthwise direction of the moving plate 214 of the first insertion device 210 may be parallel to a minor axis direction of the elliptical shape of the first elastic part 160. Thus, the first insertion device 210 may be moved in the minor axis direction and a major axis direction according to the elliptical shape of the first elastic part 160. In this case, since the first elastic part 160 has an elliptical shape, the first insertion device 210 may have a wider range of movement in the major axis direction than in the minor axis direction.

Accordingly, in the first insertion device 210, the insert 212 may move in the lengthwise direction of the moving plate 214, and the moving plate 214 may move in the major axis direction of the first elastic part 160. The range of movement of the insert 212 may be set by the length of the insertion hole MH of the moving plate 214 and the area of the first elastic part 160.

Although there will be no description of the second insertion device 220 and the third insertion device 230, they may have the same construction as the first insertion device 210.

Meanwhile, the moving plate 214 of the first insertion device 210 may have a different shape from the moving plate 214 illustrated in FIG. 3 while the insert 212 remains the same.

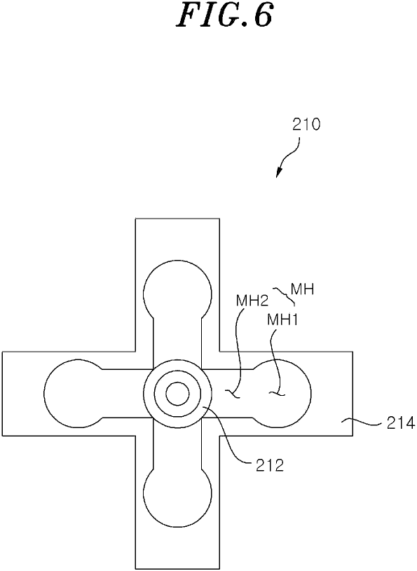

For example, as illustrated in FIG. 6, the moving plate 214 may have a cross shape, and a cross-shaped insertion hole MH may be formed inside the cross shape. Fixing hole parts MH1 may be formed on the ends of the cross shape of the insertion hole MH, and one fixing hole part MH1 may be formed at the center of the cross shape. A moving hole part MH2 may be formed between each of the fixing hole parts MH1.

The fixing hole parts MH1 may be larger in width so as to fix the insert 212, and the moving hole part MH2 may be smaller in width so as to allow the insert 212 to move.

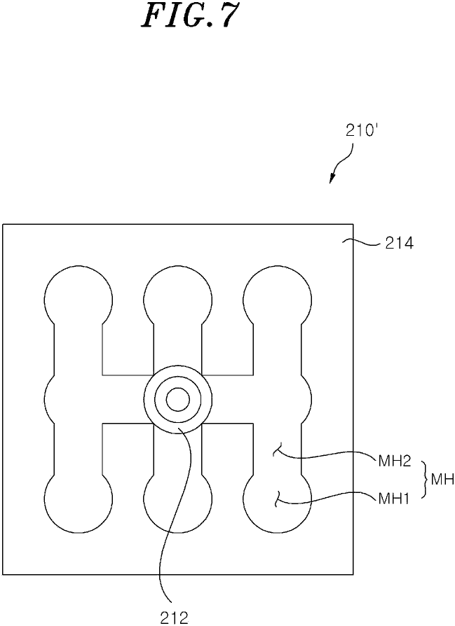

As another example, as illustrated in FIG. 7, the moving plate 214 may have a rectangular shape and include an insertion hole MH having a six-pronged path therein.

The six-pronged insertion hole MH has three paths (in the lengthwise direction shown in FIG. 7) of a predetermined length in one direction that are formed by the moving hole part MH2, and the three paths are placed side by side in parallel with each other so as to be spaced apart from each other. The insertion hole part MH may be shaped in such a way that the centers of the three paths are connected (in the horizontal direction shown in FIG. 7).

Accordingly, the fixing hole parts MH1 may be respectively formed on ends of the six prongs, and the fixing hole parts MH1 may be respectively formed at the centers of the three paths. Thus, a total of nine fixing hole parts MH1 may be formed.

As described above, since the insertion hole MH is formed in the moving plate 214, the insert 212 may be moved to where it is supposed to be, depending on the shape of the insertion hole MH.

The protection device for transoral surgery according to the embodiment of the present disclosure is a device for protection when performing thyroid surgery through a vestibular approach. Along with this, in recent transoral surgeries, surgery can be performed through a sublingual approach. In the sublingual approach, surgery is performed by selecting a sublingual portion as a region to be incised in the oral cavity, incising the corresponding portion, and inserting the trocar 300.

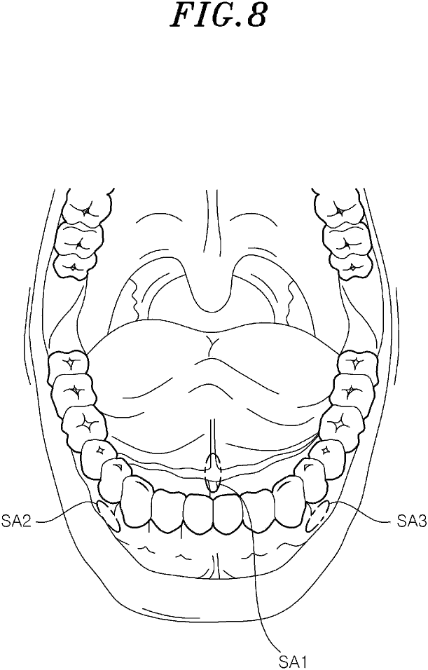

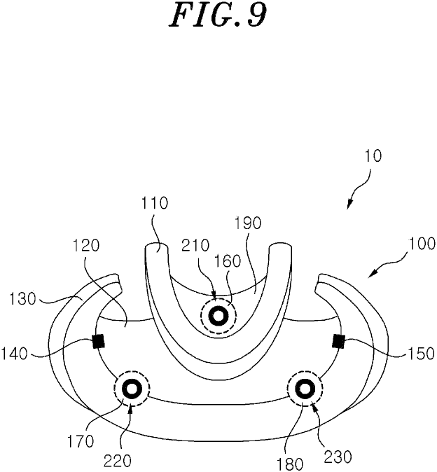

That is, as illustrated in FIG. 8, the second incision area SA2 and the third incision area SA3 are identical to those in the one embodiment, except that the first incision area SA1 is situated on an inner side of the row of lower teeth. Accordingly, a protection device for transoral surgery according to another embodiment of the present disclosure may be configured as illustrated in FIG. 9.

The protection device 10 for transoral surgery according to another embodiment of the present disclosure includes a main body 100, a first insertion device 210, a second insertion device 220, and a third insertion device 230. In describing the protection device for transoral surgery according to another embodiment of the present disclosure, any redundancies with the one embodiment of the present disclosure will be omitted.

As in the one embodiment, the main body 100 is fitted into the oral cavity of a patient and secure a surgical space for transoral surgery. The main body 100 may include a tooth coupling part 110, an extension part 120, an expansion part 130, a first safety part 140, a second safety part 150, a first elastic part 160, a second elastic part 170, a third elastic part 180, and a tooth row extension part 190.

Although another configuration of the main body 100 is identical to that of the one embodiment, the main body 100 further includes a tooth row extension part 190. The tooth row extension part 190 may be formed on the inside of the tooth coupling part 110, and may be shaped in such a way as to connect to the U-shaped inside. In this instance, the tooth row extension part 190 may be curved where it is not joined to the tooth coupling part 110. The tooth row extension part 190 may be shaped to correspond to the shape of the oral cavity since it makes contact with a sublingual portion in the oral cavity.

Unlike the one embodiment, the first elastic part 160 may be placed in the tooth row extension part 190. Also, the first insertion device 210 may be placed inside the first elastic part 160. No other component may be placed between the second elastic part 170 and the third elastic part 180.

As the first elastic part 160 and the first insertion device 210 are placed in the tooth row extension part 190, the first insertion device 210 may be placed in a position corresponding to the first incision area SA1 situated in a sublingual portion.

Accordingly, transoral surgery may be performed according to a sublingual approach by using the protection device 10 for transoral surgery according to another embodiment of the present disclosure.

As described above, the embodiments of the present disclosure have been described as specific examples, but these are only examples, and the present disclosure is not limited thereto, and should be construed as having the widest scope according to the technical spirit disclosed in the present specification. A person skilled in the art may combine/substitute the disclosed embodiments to implement a pattern of a shape that is not disclosed, but it also does not depart from the scope of the present disclosure. In addition, those skilled in the art can easily change or modify the disclosed embodiments based on the present specification, and it is clear that such changes or modifications also belong to the scope of the present disclosure.

What is claimed is:

1. A protection device for transoral surgery comprising:
a main body configured to be fitted into an oral cavity of a human body and to expand a mouth vestibule between teeth and a lip of the human body; and
at least one insertion device provided to the main body and having a through hole formed therethrough to enable insertion of a trocar required for a surgery,
wherein the main body includes:
a tooth coupling part configured to be fitted on the teeth of the human body;
an expansion part to be disposed on an outside of the tooth coupling part and configured to expand the mouth vestibule to allow the teeth and the lip to be spaced apart from each other; and
an extension part configured to connect the tooth coupling part and the expansion part.

2. The protection device for transoral surgery of claim 1, wherein the at least one insertion device includes a plurality of insertion devices, and one or more of the plurality of insertion devices are disposed on the outside of the tooth coupling part.

3. The protection device for transoral surgery of claim 1, wherein the at least one insertion device includes a plurality of insertion devices, and one of the plurality of insertion devices is disposed on an inside of the tooth coupling part.

4. The protection device for transoral surgery of claim 3, wherein the main body further includes a tooth row extension part having a predetermined width and formed on the inside of the tooth coupling part,
wherein one of the plurality of insertion devices is disposed in the tooth row extension part.

5. The protection device for transoral surgery of claim 1, wherein the main body further includes:
at least one elastic part having a predetermined width, which is formed of a softer material than a material of the tooth coupling part and has a predetermined level of elasticity,
wherein the at least one insertion device is disposed in the at least one elastic part, respectively.

6. The protection device for transoral surgery of claim 1, wherein the main body further includes:
a first safety part and a second safety part which are placed on the expansion part, and formed of a rigid material that is harder than a material of the expansion part,
wherein the least one insertion device is placed between the first safety part and the second safety part.

7. The protection device for transoral surgery of claim 1, wherein the at least one insertion device comprises an insert, and
wherein the insert includes:
a cylindrical insertion housing having a hollow therein; and
an insertion moving object having the through hole and disposed in the hollow of the insertion housing, at least a portion of the insertion moving object having a spherical shape so that an orientation of the through hole is changed.

8. The protection device for transoral surgery of claim 7, wherein a height of the insertion housing is smaller than a diameter of the insertion moving object.

9. The protection device for transoral surgery of claim 7, wherein the at least one insertion device further comprises:
a moving plate that has a shape of a plate with a predetermine length, and has therein an insertion hole of a predetermined length formed so that the insert moves in a lengthwise direction of the plate shape.

10. The protection device for transoral surgery of claim 9, wherein the insertion hole formed in the moving plate comprises:
a plurality of fixing hole parts of a predetermined width so as to fix the insert; and
a moving hole part that extends between the plurality of fixing hole parts and is smaller in width than the fixing hole parts.

11. The protection device for transoral surgery of claim 9, further comprising:
at least one elastic part whose planar shape is elliptical, and which is formed of a softer material than a material of the tooth coupling part and has a predetermined level of elasticity,
wherein the at least one insertion device is placed in the at least one elastic part, respectively, so that the lengthwise direction of the moving plate is parallel to a minor axis direction of the elliptical shape of the at least one elastic part.
